(19) United States
(12) Patent Application Publication
(10) Pub. No.: US 2010/0092398 A1
(43) Pub. Date: Apr. 15, 2010

(54) ANTIMICROBIAL COMPOSITIONS AND APPLICATIONS THEREFORE

(76) Inventor: Max Reynolds, Kuraby (AU)

Correspondence Address: OBLON, SPIVAK, MCCLELLAND MAIER & NEUSTADT, L.L.P., 1940 DUKE STREET, ALEXANDRIA, VA, 22314, US

(21) Appl. No.: 12/640,785

(22) Filed: Dec. 17, 2009

Related U.S. Application Data
(63) Continuation-in-part of application No. 11/239,014, filed on Sep. 30, 2005, now abandoned.
(60) Provisional application No. 60/688,354, filed on Jun. 8, 2005.
(60) Provisional application No. 60/614,329, filed on Sep. 30, 2004.

(51) Int. Cl. A61K 31/045; A61P 31/00; A61K 9/12

(52) U.S. Cl. 424/43; 514/729

(57) ABSTRACT

A composition derived from an essential oil obtained from one or more plants of the Melaleuca family in which at least 80% of the monoterpene content of the oil has been removed. Practical uses of the composition including antimicrobial, antiviral and therapeutic applications. Medical devices to which the composition has been applied and/or absorbed thereon.

### CROSS-REFERENCE TO RELATED APPLICATIONS

This application is a continuation in part of U.S. Ser. No. 11/239,014 which claims the benefit of U.S. provisional Patent application 60/614,329 filed on 30 Sep. 2004 and U.S. Provisional Patent Application 60/688,354 filed on 8 Jun. 2005, the disclosures of which are incorporated herein by reference.

### FIELD OF THE INVENTION

The present invention relates to a composition derived from an essential oil obtained from one or more plants of the Melaleuca family. In particular, the present invention also relates to therapeutic applications of the composition. The present invention in a particular embodiment relates to medical devices to which the composition has been applied and/or absorbed thereon.

### BACKGROUND OF THE INVENTION

The present invention will be described with particular reference to a composition derived from Melaleuca alternifolia. However, it will be appreciated that compositions of the invention may also be sourced from other Melaleuca species and no limitation is intended thereby. Other suitable sources of compositions of the present invention include but are not limited to M. linarifolia, M. dissitiflora, M. bracteata, M. cricifolia and M. quinquinervia.

Essential oils are complex mixtures of volatile oils produced by plants and are responsible for the odor of many plants. The essential oil, once produced, is either released to the environment or stored in oil cells for later use. Essential oils stored in the wood of plants serves to deter microorganisms and insects from attack.

The essential oil obtained from the steam distillation of the stems and leaves of Melaleuca alternifolia is known as tea tree or Melaleuca oil. Tea tree oil is well characterized and is described at page 1622 of the Thirteenth edition of the Merck Index 2001, which entry is incorporated herein by reference. Tea tree oil has been allocated CAS number 68647-73-4, incorporated by reference.

Essential oils of Melaleuca contain large amounts of terpenes. Terpenes are classified according to the number of units of the basic structure methylbuta-1,3-diene or isoprene, which make up the terpene. Monoterpenes contain two isoprene units and have the chemical formula C₁₀H₁₆. Monoterpenes may be acyclic such as myrcene and ocimene or cyclic such as limonene. Commercially available Melaleuca oil can comprise up to about 50% monoterpenes and typically between about 30% and 40%. Monoterpenes found in Melaleuca oil include ∝-pinene, sabinene, γ-terpinene, ∝-terpinene and limonene with γ-terpinene being the predominant monoterpene at levels in the order of 17-20%.

Essential oils typically also contain sesquiterpenes. Sesquiterpenes contain three isoprene units and have the general formula C₁₅H₂₄ and are generally found in much lower quantities than the monoterpenes. For example, Melaleuca oil typically contains about 4 to 8% sesquiterpenes.

Another class of compounds commonly found in essential oils are known as oxygenates or oxygenated terpenes. These compounds have a terpene skeleton and an oxygen containing functional group. Examples are aldehydes, phenol alcohols, carboxylic acids, ketones and esters. Terpinen-4-ol, having the formula, C₁₀H₁₈O is a major constituent of Melaleuca oil and can constitute up to 48% of the oil. 1,8 cineole is another oxygenated terpene found in Melaleuca oil. Terpinen-4-ol is considered to be the major active constituent of Melaleuca oil. However, other oxygenated products and the monoterpenes are also believed to have some antimicrobial activity.

Oxygenated terpenes are sometimes loosely referred to as monoterpenes, whereas others use the term monoterpene to refer only to compounds having the formula C₁₀H₁₆. As the latter is considered to be the more accurate nomenclature, this is the nomenclature that will be used in the present specification and claims. Monoterpenoid is another term that is understood to include the monoterpenes and other related compounds having the monoterpene skeleton.

The actual content of compounds found in essential oils, including Melaleuca oil is subject to variation depending upon the original content of the source plant and variations in extraction methods. That a conventionally steam distilled Melaleuca oil may contain varying amounts of monoterpenes is well accepted by those of skill in the art. Such variation occurs with changes in season, weather and from tree to tree. Commercial producers minimize variation by generous batch sizes. Such variations have not been reported to cause any significant variation in properties of Melaleuca oil.

Commercially available Melaleuca or tea tree oil must comply with International standards; ISO 4730:2004 for Oil of Melaleuca (tea tree oil). There is also an Australian standard AS 2782-1997. Both of these standards, including the physical and chemical parameters they require or describe, are incorporated by reference. In order to comply with these standards, the essential oil must be obtained by steam distillation of the foliage and terminal branches of a Melaleuca plant that contains levels of 15 compounds in the compositional limits set by the standards. High quality oil is considered to be an oil having high levels of terpinen-4-ol and low levels of 1,8-cineole. The source plant is not limited to M. Alternifolia but can be obtained from other species such as M. dissitiflora and M. linarifolia.

The standards require the oil to be analysed by gas chromatography using a flame ionization detector. The proportions of components, in % are indicated by the integrator. In the present specification and claims, the % proportions of components of the oil will also represent the proportion indicated by an integrator.

The contents of both the International standard ISO 4730-2004 and Australian Standard AS 2782-1997 are incorporated herein by reference.

The main constituents of tea tree oil from the International (ISO 4730:2004) and Australian (AS 2782-1997) standards, together with an indication of the typical amounts found in a commercial tea tree oil are shown in the following Table 1. These standards are similar but contain small differences in the maximum values for some components.

TABLE 1

| | | % max | % max | |
|---|---|---|---|---|
| | | ISO | AS 2782- | |
| Component | % min | 4730:2004 | 1997 | Typical % |
| terpinolene* | 1.5 | 5 | 5 | 3.4 |
| 1,8-cineole (eucalyptol)^{#} | Tr | 15 | 15 | 2.6 |
| ∝-terpinene* | 5 | 13 | 13 | 9.7 |
| γ-terpinene* | 10 | 28 | 28 | 19.7 |
| p-cymene | 0.5 | 8 | 12 | 1.3 |
| terpinene-4-ol^{#} | 30 | 48 | no max. | 39.6 |
| ∝-terpineol^{#} | 1.5 | 8 | 8 | 2.9 |
| limonene* | 0.5 | 1.5 | 4 | 3.4 |
| sabinene* | tr | 3.5 | 3.5 | 0.4 |
| aromadendrene+ | tr | 7 | 3 | 1.7 |
| δ-cadinene+ | tr. | 3 | 8 | 1.4 |
| globuol^{#} | tr. | 1 | 3 | 0.5 |
| viridiflorol^{#} | tr. | 1 | 1.5 | 1.3 |
| ∝-pinene* | 1 | 6 | 6 | 2.5 |
| ledene+ (syn. viridiflorene) | tr. | 3 | | 1.0 |
| total monoterpenes | 18 | 59.5 | | 39.1 |

*monoterpenes
+sesquiterpenes
^{#}oxygenated terpenes

Terpenes contain double bonds, which are susceptible to oxidation. It is believed that the capacity to generate activated oxygen intermediates may be responsible for their antimicrobial activity. On the other hand, this susceptibility to oxidation results in instability. Terpenes, particularly monoterpenes, are primarily saturated hydrocarbons, which are vulnerable to oxidation by oxygen in the environment surrounding the monoterpenes. The attack occurs in the region of the C-C double bonds of the terpene molecule. Such instability typically leads to discoloration, odor and premature loss of the proactive sites and also accounts for some of the observed heat sensitivity and chemical reactivity of the essential oils. A further disadvantage is that some of these oxidation products may be irritating or even allergenic.

Relatively crude Melaleuca extracts obtained by steam distillation have been recognized to exhibit antifungal properties, but to have only limited antimicrobial properties, see U.S. Pat. No. 7,258,876. Terpinen-4-ol has been identified as a putative high potency component of Melaleuca oil, U.S. Pat. No. 6,514,539. U.S. Pat. No. 6,514,539 describes a Melaleuca Alternifolia fraction having enhanced levels of terpinene-4-ol. Although this fraction is described as “high potency”, its action against E. Coli is comparative to that of Melaleuca oil and is only about twice as effective against Staphylococcus aureus.

The present inventor has surprisingly and unexpectedly discovered that a composition derived from Melaleuca essential oil, whereby a major portion of the monoterpene content has been removed, not only exhibits improved stability but also retains and, in some cases, increases its antiseptic and antimicrobial properties. Such a composition may be derived from conventional Melaleuca oil meeting the Australian or International standards, but is processed, extracted, distilled, refined, or otherwise treated to remove monoterpenes.

The present inventor has further observed that a preferred composition of the present invention exhibits improved antimicrobial properties when compared with conventional Melaleuca oil. Further still, the present inventor has discovered that whilst conventional Melaleuca oil is suitable only for topical administration in view of its toxicity when ingested, that a preferred composition of the invention may be considered safe for oral administration. Toxic effects, which may be experienced after ingestion of Melaleuca oil include seizures, coma and respiratory depression.

### BRIEF DESCRIPTION OF THE INVENTION

It is therefore an object of the present invention to provide a composition, which may at least partially overcome the above disadvantages or provide the public with a useful or commercial choice. It is also an object of the present invention to provide methods of treatment for conditions associated with, or caused by, infectious agents. A further object is to provide medical devices with biocidal properties.

According to a first broad form of the invention, there is provided an essential oil extract produced by treating a conventional Melaleuca oil to remove at least 80% of the total monoterpene content such that the extract contains 10% or less than 10% monoterpenes.

### DETAILED DESCRIPTION OF THE PREFERRED EMBODIMENTS

The term “monoterpene” refers to compounds derived from an isoprene unit that has the formula C₁₀H₁₆. Monoterpenes found in the essential oil from Melaleuca Alternifolia include ∝-pinene, ∝-thujene, β-pinene, sabinene, ∝-phellandrene, ∝-terpinene, limonene, γ-terpinene, β-phellandrene, γ-terpinene and terpinolene.

The term “conventional Melaleuca oil” refers to oil extracted from a Melaleuca plant that complies with Australian Standard AS 2782-1997 incorporated herein by reference. Preferably, the conventional Melaleuca is extracted from Melaleuca alternifolia.

As discussed above, essential oils derived from Melaleuca species comprise a monoterpene fraction, an oxygenate fraction and a sesquiterpene fraction, although it will be appreciated that different species may contain different relative amounts of each fraction. Of these fractions, the monoterpenes are generally the most volatile and have the lowest molecular weight. Thus, they may be removed by techniques known to those of skill in the art including vacuum low temperature techniques, such as inert gas flushed distillation; fractional distillation, molecular weight separation techniques including chromatographic techniques and selective solvent extraction techniques. Preferably, the monoterpenes are removed under reduced pressure and at a temperature that does not exceed 50° C., preferably 40° C. Suitable chromatographic techniques have been described in U.S. Pat. No. 4,605,783 and by Hayashi, et al., Bulletin of the Chemical Society of Japan, vol 42, 3026-3028 (1969), both of which are incorporated by reference). The use of fractional distillation of essential oils is well known for obtaining fractions of different boiling points. The monoterpenes found in Melaleuca oil have boiling points ranging from about 155° C. for ∝-pinene to about 185° C. for terpinolene. Terpinen-4-ol has a boiling point of 212° C. The sesquiterpenes aromadendrene and alloaromadendrene have boiling points of 257° C. to 258° C.

Generally between about 80% and about 99% of the monoterpenes are removed, typically between about 90% and about 99%. Preferably, the extract contains less than 10%, 7.5%, 5%, 4%, 3%, 2% and most preferably less than 1% or 0.5% monoterpenes. Where there are less than 1% monoterpenes, the levels of any one or more of the monoterpenes may fall below the detectable limit of the gas chromatograph used for analysis. This well understood by a person of skill in the art.

It may be seen that the combined minimum and maximum contents for all of the monoterpenes in Melaleuca oil defined by both the Australian and International standards provides a range of monoterpene content from 18% to 59.5%. It may therefore be clearly appreciated that the composition of the present invention that has a maximum total monoterpene content of 10% falls well outside the standard range and therefore cannot be considered to have the same chemical profile as conventional Melaleuca or tea tree oil.

It will be appreciated that the exact amount of monoterpenes required to be removed to provide a maximum content of 10% may vary, depending upon the monoterpene content of the parent conventional oil. This amount may be readily calculated by a skilled person. For example, a typical Melaleuca oil contains about 40% monoterpenes. Removal of just over 80% i.e. 83.5% of the monoterpenes yields a final extract having a total remaining monoterpene content of 10%. It will also be appreciated that by removing a significant proportion of the total content of the oil, the compositional profile of other non-monoterpene components will be changed.

A preferred composition of the invention is derived from the essential oil of Melaleuca alternifolia and typically comprises from between about 40% to about 70%, preferably between about 50% to about 65% most preferably between about 60% to about 65% terpinen-4-ol and between about 8 to about 30%, preferably between about 8% to about 25% sesquiterpenes. These ranges include all intermediate subranges and values. The sesquiterpene fraction may include aromadendrene, viridiflorene, delta cadinene, globulol and/or viridiflorol. Compositions comprising some or all of the components, described herein, of Melaleuca oil from which at least 80% of the monoterpenes in conventional Melaleuca oil have been removed are also contemplated.

The composition of the invention may find particular application as an antimicrobial agent. Thus, according to a further broad form of the invention, there is provided an antimicrobial composition derived from an essential oil of Melaleuca, wherein at least about 80% of the monoterpene content of the oil has been removed.

The antimicrobial composition may find application as an antibacterial, antiprotozoan, antifungal and/or antiviral agent. Typically, the composition is effective against a broad range of micro-organisms including E. coli, S. aureus, P. aeruginosa, C albicans, S. epidermidis, Penicillium ssp, Cladosporium, A. niger, A. fumigatus, P. expansum, S. chartarum, Alteraria, Aspergillus, Fusarium, B. subtilis, B. cereus, C. perfringens, K. pneumoniae, L. lactis, M. smegmatis, S. marcescens, S. pyogenes, A. viridans, E. aerogenes, S. liquefaciens, P. vulgaris, S. enteridis, P. mirabilis, S. abaetetuba, L. monocytogenes, N. gonorrhoeae, Legionella, M. gordanoae and M. catarrhalis and viruses including coronavirus, rotavirus, adenovirus, herpes simplex, papillovirus, rhinovirus, hepatitis B and A, enterovirus and respiratory viruses such as influenza and parainfluenza virus.

The composition may be used as a disinfectant and/or anti-mold agent for hard surfaces such as those typically found in homes like kitchens, bathrooms, tiles, walls, floors, chrome, glass, smooth vinyl, any plastic, plastified wood, table top, sinks, cooker tops, dishes, sanitary fittings such as sinks, showers, shower curtains, wash basins and the like. Hard-surfaces also include household appliances including but not limited to, refrigerators, freezers, washing machines, automatic dryers, ovens, microwave ovens, dishwashers and surfaces found in hospitals, restaurants, hotels, means of public transport, public bathes and pools, commercial and public laundries and the like are included herein.

The composition may further comprise optional ingredients such as a solvent, surfactant, chelating agent, fragrance, carrier, diluent, one or more other essential oils and the like. Such optional ingredients are known to those of skill in the formulation arts.

The composition of the invention may also be used to control airborne infectious agents. In this case, the composition may be formulated together with a suitable propellant. A preferred propellent is nitrogen in which droplets of the composition are dispersed.

Infectious agents that may spread via airborne droplets include chickenpox, common cold, diphtheria, Haemophilus influenza type b (Hib), influenza, measles, meningitis (bacterial), meningococcal disease, mumps, parvovirus infection (human parvovirus infection, parvovirus B19 infection, slapped cheek, slapped face, erythema infection, fifth disease), Pneumococcal pneumonia, rubella, streptococcal sore throat, tuberculosis and whooping cough (pertussis). The composition of the present invention may be used to control such airborne infectious agents. The compositions may find particular application in otherwise sterile environments such as operating theatres, isolation wards and the like.

According to a further broad form of the invention there is provided an anti-microbial aerosol composition comprising the composition of the first broad form dispersed in a propellent.

According to a further broad form of the invention there is provided a method of controlling airborne infectious agents in an atmospheric environment, the method comprising dispersing an effective amount of the aerosol of the previous embodiment into the environment.

The composition may also find use as a topical anti-microbial and/or anti-parasitic agent for human or animal use, including livestock animals such as bovines, goats, sheep, fowl, including chickens, geese, and ducks, fish, and other domesticated or domestic animals, including pets such as dogs, cats, birds and aquarium fish. Exemplary applications include cosmetics, hand sanitizers, antiseptic scrubs or washes as well as flea and lice shampoos.

The composition may also be used to treat ulcers, including those caused by Group A streptococcus and Staphylococcus aureus including MRSA and other antibiotic-resistant microrganisms; fungal infections such as ringworm or athlete's food, insect or parasite bites or infestations, burns, cuts, abrasions, wounds, acne, diabetic gangrene, secondary microbial infections, for use in subjects having a compromised epidermal barrier or mucosal membrane, as well as for palliative care of cancer patients. Topical preparations may contain a lipophilic carrier (e.g, glycerin, vegetable oils, fish oils, including those containing high concentrations of omega-3 oils, petroleum jelly or other petroleum derivatives), a water or fat-soluble antioxidant, such as a retinol (e.g., vitamin A) or carotene (e.g., beta-carotene), vitamin E, vitamin C (e.g., ascorbic acid or ascorbyl palmitate), an emulsifier, and/or an aqueous phase, as well as other antimicrobial agents or antiseptics, including hydrogen peroxide or iodine-containing products, in addition to the composition of the invention. The composition of the invention may be used in its solid (e.g., in soaps, powders, and bath salts), semi-solid (e.g., ointments, salves), liquid (e.g., lotions, body washes, shampoos, liquid soaps, emulsified aqueous solutions, liquid cleaners or disinfectants) or aerosol forms.

The concentration of the composition of the invention to be incorporated into such products may be selected according to the desired use, e.g., the minimal inhibitory or cidal concentration for a particular organism, and may range between 0.01%. 1%, 5%, 10%, 25%, 50%, 75% to 100% of the topical composition, including all intermediate values and subranges.

The inventor has further surprisingly and unexpectedly discovered that the composition of the present invention possesses topical analgesic and anti-inflammatory properties.

According to a further broad form of the invention there is provided a topical therapeutic composition comprising a composition of the first broad form.

The topical composition may be in any suitable form including an aerosol, preferably a nitrogen aerosol, a cream, gel or oil. For wound treatment, the aerosol typically has a particle size of between about 5, 7.5, 10, 12.5, 15 to about 20, preferably about 10 micron and for an analgesic between about 40, 45, 50, 55, 57.5 to about 60 micron. These ranges include all intermediate subranges and values. Typical carriers for topical administration include water, alcohol, silicone, other essential oils, oils, wax and gels. Typically, for analgesic or anti-inflammatory use, a topical composition comprises between about 1% to about 5% of the inventive composition, including any intermediate values, such as 1.5%, 2%, 3%, 4%, 4.5% and 4.75%.

The anti-inflammatory properties of the composition may make it suitable for use in the external treatment of painful muscles, tendons, skin irritations, gout, periodontal disease, gingivitis and the like. The composition may also be suitable for internal use for the treatment of conditions associated with an inflammatory response such as colon, bowel, lung, throat and nose infections.

The topical compositions may also be administered in the form of wound dressings, transdermal patches and the like. Typically wound dressings are impregnated with a composition of the invention at a concentration of active agents of between about 1,000 to about 5,000 ppm and all intermediate subranges and values.

A still further use of a composition of the invention is as an antibiotic or anti-viral agent for internal human or animal use. It will be appreciated by those of skill in the art that conventional essential oils containing monoterpenes are considered unsuitable for oral ingestion. Cases of poisoning have been reported after accidental ingestion of commercial Melaleuca oil.

According to a further broad form of the invention, there is provided a pharmaceutical composition for treating an infection in an animal, the composition comprising an effective amount of the composition of the first broad form together with a pharmaceutically effective carrier diluent, excipient and/or adjuvant.

According to a further broad form of the invention, there is provided a method for the treatment or prophylaxis of an infection in an animal, the method comprising administering to the animal an effective amount of the composition of the first broad form of the invention.

The infectious agent includes any agent that may be responsible for, or contribute to, a health condition in an animal. Such infectious agents include bacteria, fungi and viruses and parasitic infectious agents such as malaria, hookworm, tapeworm, Giardia and the like.

The pharmaceutical composition may be in any suitable form. Solid form preparations include powders, tablets, dispersible granules, capsules, cachets, suppositories and ointments. Liquid form preparations include solutions, suspensions and emulsions suitable for oral ingestion or injection.

Typical dosage level may be between about 0.001, 0.01, 0.1, 1.0, 10.0, 25.0. 50.0, 75.0 and about 100 mg/kg body weight per day, preferably between about 0.5 and about 75 mg/kg body weight per day, which ranges include all intermediate subranges and values. Typically, the pharmaceutical compositions of this invention will be administered from about 1 to about 5 times per day or alternatively, preferably on a daily basis.

As the skilled artisan will appreciate, lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination and the severity of the condition.

In a particularly preferred form of the invention, the pharmaceutical composition is administered to the lung by the pulmonary route. Such administration may be suitable for the treatment of diseases caused by upper respiratory and lung infections such as tuberculosis, bacterial and viral pneumonia and influenza. The composition may be administered using known facial vaporizers. Conveniently, the composition is formulated as an aerosol. Preferably, the aerosol administers particles having a size of up to about 10 microns. Preferably, the aerosol is formulated using nitrogen as a propellant.

The composition of the present invention may also be used in association with medical devices. The device may be treated with the composition in any suitable manner such as by coating, absorption and impregnation, or in the case of thermoplastic materials, co-extruded therewith. The medical devices may be external devices such as catheters, urinary bags and any suitable container that may in use come into contact with an infectious agent. Internal devices may include stents and implants such as breast implants. The surface of a plastic medical device may be pre-treated prior to coating to facilitate absorption on, or adhesion thereto. Such pre-treatment may include surface activation by methods such as corona or plasma treatment, or by sonically disrupting the plastic surface. Alternatively, or in addition to, the composition may be formulated to facilitate attachment to a plastic surface. For example, in the case of a silicone breast implant, the compositions may be formulated in a silicone based carrier or diluent.

The composition of the present invention may also be used as a fabric and/or garment biocide. Suitable fabrics include natural and synthetic fabrics and also fabrics formed from or including cellulosic materials. It is often desirable for sporting or active wear garments to be treated with a biocide, so as to control odor releasing bacteria. Biocidal fabrics find particular application in hospitals or other healthcare situations. The composition of the invention may be used in association with gloves, head covers, gowns, boots, head bands and the like. A particular application is for surgical masks. Typically, surgical masks may be treated by spraying an aerosol onto both sides of the mask.

According to a further broad form of the invention there is provided a fabric to which a composition of the first broad form has been applied.

The composition maybe applied by any suitable means such as spraying, dipping painting or coating.

### Examples

### Example 1

A composition of the present invention was prepared by removing essentially all the monoterpene fraction from Melaleuca alternifolia essential oil. The composition of the oils before and after removal of the monoterpene fraction was analysed by gas chromatography and described by gas chromatograms of Melaleuca oil before and after monoterpene removal.

Table 2 shows a profile of a typical composition of the present invention following monoterpene removal (referred to as MAC) and for comparison the profile of the Melaleuca oil (TTO, tea tree oil) before removal of the monoterpenes.

TABLE 2

| Melaleuca oil prior to | | | | | | |
|---|---|---|---|---|---|---|
| monoterpene removal | | | Melaleuca oil after monoterpene removal | | | |
| | TTO | | | Extract | | |
| TTO | R. | | Extract | R. | | Compound |
| Peak # | Time/min | Area % | Peak # | Time/min | Area % | (ISO 4730 Range %) |
| 1 | 3.106 | 2.56 | 1 | 3.108 | 0.05 | ∝-pinene* (1-6) |
| 2 | 3.892 | 0.62 | 2 | 3.982 | 0.05 | β-pinene* |
| 3 | 4.123 | 0.28 | | | | sabinene* (tr-3.5) |
| 4 | 4.621 | 0.67 | | | | myrcene* |
| 5 | 4.682 | 0.41 | 3 | 4.623 | 0.03 | phellandrene* |
| 6 | 4.878 | 7.92 | 4 | 4.878 | 0.04 | ∝-terpinene* (5-13) |
| 7 | 5.137 | 0.74 | 5 | 5.139 | 0.25 | limonene* (0.5-1.5) |
| 8 | 5.258 | 3.01 | 6 | 5.259 | 1.12 | 1,8-cineole (tr-15) |
| 9 | 5.815 | 16.65 | 7 | 5.811 | 0.57 | γ-terpinene* (10-28) |
| 1-9 | <6 min | 32.86 | 1-7 | <6 min | 2.11 | subtotal of peaks with |
| | | | | | | R. Time <6 min. |
| 10 | 6.191 | 2.65 | 8 | 6.197 | 9.06 | p-cymene (0.5-8) |
| 11 | 6.339 | 3.05 | 9 | 6.390 | 0.19 | terpinolene* (1.5-5) |
| | | | 10 | 6.649 | 0.02 | unidentified |
| 12 | 6.915 | 0.02 | | | | unidentified |
| 13 | 8.212 | 0.05 | | | | unidentified |
| 14 | 10.195 | 0.04 | 11 | 10.200 | 0.07 | ∝-copaene + |
| | | | 12 | 10.325 | 0.13 | unidentified |
| 15 | 10.473 | 0.03 | 13 | 10.481 | 0.04 | isoledene + |
| 16 | 10.925 | 0.02 | 14 | 10.935 | 0.03 | unidentified |
| 17 | 11.150 | 0.07 | 15 | 11.155 | 0.13 | ∝-cubebene + |
| 18 | 12.275 | 0.25 | 16 | 12.279 | 0.26 | ∝-gurjunene |
| 19 | 12.798 | 0.05 | 17 | 12.797 | 0.06 | linalool |
| 20 | 13.233 | 0.32 | 18 | 13.239 | 0.26 | trans, p-menth-2-en- |
| | | | | | | 1-ol |
| 21 | 14.293 | 0.02 | 19 | 13.945 | 0.67 | n-caproic acid vinyl |
| | | | | | | ester |
| | | | 20 | 14.308 | 0.03 | unidentified |
| 22 | 14.467 | 0.30 | 21 | 14.471 | 0.15 | bicyclo [7.2.0] |
| | | | | | | undec-4-ene |
| 23 | 14.661 | 49.06 | 22 | 14.668 | 59.29 | terpinene-4-ol (30-48) |
| 24 | 14.849 | 1.06 | 23 | 14.858 | 1.78 | aromadendrene + (tr- |
| | | | | | | 3) |
| 25 | 14.982 | 0.03 | 24 | 14.977 | 0.06 | unidentified |
| 26 | 15.133 | 0.09 | 25 | 15.136 | 0.16 | ∝-selinene + |
| 27 | 15.565 | 0.24 | 26 | 15.572 | 0.25 | cyclohexen-1-ol |
| 28 | 15.726 | 0.10 | | | | unidentified |
| 29 | 16.182 | 0.51 | 27 | 16.789 | 0.89 | alloromamendrene + |
| 30 | 16.803 | 0.20 | 28 | 16.968 | 0.05 | cadin-1(6), 4-diene |
| 31 | 17.091 | 0.04 | 29 | 17.102 | 0.02 | unidentified |
| 32 | 17.913 | 0.10 | 30 | 17.911 | 0.23 | β-bisabolene + |
| 33 | 18.161 | 0.88 | 31 | 18.172 | 0.81 | viridiflorene (ledene) |
| | | | | | | (tr-3) |
| 34 | 18.292 | 3.97 | 32 | 18.306 | 5.74 | ∝-terpineol |
| | | | 33 | 18.531 | 4.12 | cis-β-terpineol |
| 35 | 18.832 | 0.06 | | | | unidentified |
| 36 | 19.027 | 0.04 | 34 | 19.038 | 0.12 | isosativene + |
| | | | 35 | 19.257 | 0.08 | unidentified |
| 37 | 19.239 | 0.03 | | | | unidentified |
| 38 | 19.392 | 0.08 | | | | muurolene + |
| 39 | 19.642 | 1.30 | 36 | 19.402 | 0.17 | bicyclogermacrene + |
| 40 | 20.328 | 0.08 | 37 | 20.198 | 0.14 | unidentified |
| | | | 38 | 20.325 | 0.09 | unidentified |
| 41 | 20.757 | 1.38 | 39 | 20.807 | 2.58 | δ-cadinene + (tr-3) |
| 42 | 21.733 | 0.12 | | | | unidentified |
| 43 | 23.926 | 0.03 | 40 | 23.944 | 0.47 | calamene + |
| | | | 41 | 24.915 | 0.65 | benzenemethanol |
| | | | 42 | 25.418 | 0.16 | unidentified |
| | | | 43 | 27.465 | 0.56 | unidentified |
| | | | 44 | 28.109 | 0.37 | unidentified |
| | | | 45 | 30.128 | 0.17 | unidentified |
| | | | 46 | 33.807 | 0.26 | ∝-limonene- |
| | | | | | | diepoxide |
| | | | 47 | 33.872 | 0.06 | unidentified |
| 44 | 34.168 | 0.08 | 48 | 34.188 | 0.16 | ∝-cedrene + |
| 45 | 34.644 | 0.15 | 49 | 34.660 | 0.37 | globulol (tr-1) |
| 46 | 35.064 | 0.06 | 50 | 35.070 | 0.18 | viridiflorol |
| | | | 51 | | | |
| 47 | 35.765 | 0.30 | 52 | 35.785 | 1.43 | sesquieneole |
| 48 | 36.538 | 0.04 | 53 | 36.551 | 0.08 | unidentified |
| 49 | 36.934 | 0.10 | 54 | 36.551 | 0.08 | spathulenol |
| | | | 55 | 37.962 | 0.22 | unidentified |
| | | | 56 | 38.645 | 1.39 | 3-hexyne-2,5-diol |
| 50 | 39.389 | 0.10 | 57 | 39.393 | | 1,3-dioxolane |
| | | | 58 | 40.395 | 0.08 | unidentified |
| | | | 59 | 41.555 | 0.33 | 1H-cyclo |
| | | | | | | prop[e]azulen-7-ol |
| | | | 60 | 43.134 | 0.69 | unidentified |
| | | | 61 | 45.805 | 0.92 | trans-carveol |
| | | | 62 | 46.021 | 0.11 | unidentified |
| | | | 63 | 46.393 | 0.12 | unidentified |
| | | | 64 | 47.928 | 0.06 | unidentified |
| 40-50 | >30 mins | 0.83 | 37-64 | >30 mins | 6.71 | Subtotal of peaks with a |
| | | | | | | R. Time >20 min |

monoterpenes*
sesquiterpenes+
compounds falling outside ISO 4730 are shown in italics

The area percentages in Table 2 were measured by total ion chromatography and are not directly comparable with the flame ionization detection used according to the International and Australian standards. However, they are comparable between each other and significant changes in composition of the monoterpenes can be seen. Although, the compositions are not directly comparable with flame ionization detection, the changes in the monoterpene levels are so dramatic that the inventor believes that it is reasonable to conclude that the levels of each monoterpene in the composition represented in Table 2 lies well outside of the minimum levels required by the Australian and International standards.

It may be seen that a significant proportion of all compounds of the parent tea tree oil having a retention time of less than 6 minutes have been removed. The total area % of compounds having a retention time of less than 6 minutes in the tea tree oil is 32.86% as compared to 2.11% in the extract. There has also been an increase in the concentration and number of compounds detected having a retention time of over 30 minutes. Another significant difference is the almost complete removal of γ-terpinene which dropped from 16.65% to 0.57%. The overall monoterpene content has been reduced from 35.91% to 1.18%.

### Comparison of Conventional Tea Tree Oil with Invention

| | | % max | % max | | |
|---|---|---|---|---|---|
| | % | ISO | AS 2782- | | |
| Component | min | 4730:2004 | 1997 | Typical % | Invention |
| terpinolene* | 1.5 | 5 | 5 | 3.4 | bdl-0.7 |
| 1,8-cineole | Tr | 15 | 15 | 2.6 | tr-3 |
| (eucalyptol)^{#} | | | | | |
| ∝-terpinene* | 5 | 13 | 13 | 9.7 | bdl-1.9 |
| γ-terpinene* | 10 | 28 | 28 | 19.7 | bdl-4 |
| p-cymene | 0.5 | 8 | 12 | 1.3 | 0.5-12 |
| terpinene-4-ol^{#} | 30 | 48 | no max. | 39.6 | 52-66 |
| ∝-terpineol^{#} | 1.5 | 8 | 8 | 2.9 | 4-6.5 |
| limonene* | 0.5 | 1.5 | 4 | 3.4 | bdl-0.6 |
| sabinene* | tr | 3.5 | 3.5 | 0.4 | bdl-0.5 |
| aromadendrene+ | tr | 7 | 3 | 1.7 | 1-7 |
| δ-cadinene+ | Tr. | 3 | 8 | 1.4 | 1.5-3.5 |
| globulol^{#} | Tr. | 1 | 3 | 0.5 | 0.3-2 |
| Viridiflorol^{#} | Tr. | 1 | 1.5 | 1.3 | 0.1-0.8 |
| ∝-pinene* | 1 | 6 | 6 | 2.5 | bdl-0.9 |
| ledene + (syn. | Tr. | 3 | | 1.0 | 0.5-5 |
| viridiflorene) | | | | | |
| Total | 18 | 59.5 | | 39.1 | bdl-10 |
| monoterpene* | | | | | |

*monoterpenes
+sesquiterpenes
^{#}oxygenated terpenes
bdl—below detectable limit

The ranges shown above reflect those shown in Table 2 and Examples 3 and 4 and include all intermediate values and subranges for each component. Each range may be divided by 10 to establish equal incremental content values. For example, total detectable monoterpene content which ranges from 0 to 10 may be 0, 1, 2, 3, 4, 5, 6, 7, 8, 9 or 10. It will be appreciated that the exact composition may vary between batches. It may also be appreciated that the chemical profile of the composition of the invention is substantially different to that of the standard tea tree oil.

Physical parameters of the oils were also compared with each other and to that of the Australian standard. The samples were analysed in accordance with that standard. The results are shown in Table 3.

TABLE 3

| | | | AS 2782- | |
|---|---|---|---|---|
| Physical Property | TTO | MAC | 1997 | terpinen-4-ol |
| Relative density @ 20° C. | 0.893 | 0.962* | 0.885-0.906 | 0.930-0.936 |
| (g/mL) | | | | |
| Refractive Index @ 20° C. | 1.476 | 1.488* | 1.475-1.484 | 1.4770-1.4810 |
| (°) | | | | |
| Optical rotation @ 20° C. (°) | +9.20 | +9.7 | +5-+15 | |
| Miscibility in Ethanol (2 | Clear | Clear | Clear | |
| volumes of 85% ethanol to | colorless | yellow | colorless | |
| 1 volume of sample) | solution | solution* | solution | |
| Kinematic Viscosity | 2.80 | 27.52 | | |
| (mm²s⁻¹) | | | | |
| Flash Point | 59° C. | 70° C. | 57° C. to 60° C. | |
| Boiling point | 184.7° C.-188.2° C. | 196.8° C.-209.8° C. | | 212° C. |

*outside the standard range

Clearly, the physical properties of the extract of the invention are significantly different from that of a conventional Melaleuca oil. In particular, the relative density is significantly higher and it is considerably more viscous, reflecting the significant drop in the content of relatively light monoterpenes. In practice, it has been observed that an extract of the invention has a minimum relative density of 0.9400, which effectively distinguishes it from a conventional Melaleuca oil extract. As shown by the analysis above, the extract of the invention does not fall within either the chemical or physical requirements to comply with the Australian standard for Melaleuca or tea tree oil. Thus, it is quite clear from these results that conventional Melaleuca oil and the extract of the invention are chemically, compositionally and physically distinct. Further, as the extract of the invention does not comply with the standard, it cannot be classified as tea tree oil. As will be shown below, the extract also has distinctly different microbiological activity when compared to conventional Melaleuca oil.

### Example 2

Comparative trials were conducted to assess the difference in the antimicrobial activity of conventional Melaleuca oil (TTO), MAC and purified (98.9%) Terpinene-4-ol. The experiments were carried out on 10% water solubilised solutions using Vitamin E as an emulsifier. Each strain of bacteria was treated with each of the water solubilised solutions with a concentration ranging form 10⁵ to 10⁶ bacteria/ml.

The minimal effective bacterial concentrations (i.e. the minimal concentration that killed 99.99% of the bacteria using the agar dilution technique is shown below in Table 4. The term “no level” indicates that bacteria survived exposure to relatively high levels of either 500 ppm or 1,000 ppm of the tested water-solubilised solution.

In the agar dilution technique, antimicrobial agents are incorporated into an agar medium. A diluted suspension of the test organism is then inoculated onto the medium. If the test organism is sensitive to the antibiotic the agar medium, growth will be inhibited. If the organism grows on the anti-biotic containing medium, then it is resistant to this particular antibiotic.

TABLE 4

| | TTO effective | | MAC |
|---|---|---|---|
| | concentration | terpinen-4-ol | terpinen- |
| Bacteria | Terpinen-4-ol 49% | 98.9% | 4-ol 59% |
| S. aureus | 1000 ppm | 250 ppm | 10 ppm |
| E. coli | No level (>1000 ppm) | No level (>500 ppm) | 10 ppm |
| P. | No level (>1000 ppm) | No level (>500 ppm) | 250 ppm |
| aeruginosa | | | |
| Salmonella | No level (>1000 ppm) | No level (>500 ppm) | 10 ppm |
| Listeria | No level (>1000 ppm) | No level (>500 ppm) | 10 ppm |

These results show that an extract of the invention is 50 to 100 times more active against a variety of both gram-negative and gram-positive bacteria than either tea tree oil or terpinene-4-ol. While these results also show that TTO (conventional tea tree oil) to be more effective than terpinen-4-ol. Moreover, when a correction is made for the difference in concentration of the terpinen-4-ol, the difference is less pronounced. Others have also observed a similar superior efficacy of TTO over terpinen-4-ol. On the the other hand, these results show the remarkably enhanced anti-bacterial efficacy of the extract (MAC) according to the invention.

### Example 3

Another composition of the present invention was analysed to have a composition as follows:

| | |
|---|---|
| terpinene-4-ol | 52-54% |
| ∝-terpineol | 4-6% |
| aromadendrene | 5-7% |
| viridiflorene | 3-5% |
| δ-cadinene | 1.5-3.5% |
| globulol | 1-2% |
| viridflorol | 0.3-0.6% |
| monoterpenes | bdl-10% |

The balance of the composition comprises a mixture of up to about 30 compounds in trace amounts. These compounds include other sesquiterpenes and higher molecular weight compounds.

Toxicity Studies

The acute oral toxicity of the above composition was investigated in 4 Sprague Dawley Specific Pathogen Free rats at doses of 500 and 1,000 ppm at 10 mL/kg. The experimental procedure was based on OECD guidelines for the testing of chemicals No 401.

The test was administered orally once to 2 pairs of rats at the above doses. A third group was administered the vehicle only, solubilized vitamin E solution. The equivalent volumetric dose was 10 ml/kg for all groups.

Body weights were determined immediately before test item administration and at sacrifice on day 8. All animals were observed at frequent intervals on the day of test item administration and then daily for signs of toxicity over the 7 day experimental period, at the end of the experimental period, all animals were sacrificed and subjected to a gross necroscopy examination.

No mortalities were observed during the study. No clinical abnormalities were observed for the duration of the study in any of the treated or control animals. There were no gross abnormalities noted in the major organs of any animal at necroscopy. Further analysis was carried out by gas chromatography testing of the kidneys and livers. No traces of the components of the above composition were found, indicating that all compounds are successfully excreted from the body.

Based on the results obtained from the study, the test composition, up to the highest doses tested 1,000 ppm at 10 ml/kg did not produce toxicity in the Sprague Dawley rat, in the acute oral sighting study.

These results may be compared to tea tree oil, which has been reported to have an LD₅₀ of 1.9-2.6 ml/kg.

Microbiological Testing

Plates were prepared using Tryptic Soy Agar (bacteria) and Malt Extract (yeast and mold). All micro-organisms were tested at against 10⁸ (MacFarland Standard) concentration of microorganism, etc. The results are shown below in Table 5:

TABLE 5

| | No. of tests | Average zone of |
|---|---|---|
| | carried | inhibition/mm |
| E. coli | 105 | 24 |
| S. aureus | 68 | 30 |
| P. aeruginosa | 48 | 22 |
| C. albicans | 60 | 35 |
| S. epidermidis | 30 | 38 |
| Penicillium ssp | 68 | Ng |
| Cladosporium | 89 | Ng |
| A. niger | 65 | Ng |
| A. fumigatus | 5 | Ng |
| P. expansum | 5 | Ng |
| S. chartarum | 4 | Ng |
| Alternaria | 10 | Ng |
| Aspergillus | 5 | Ng |
| Fusarium | 4 | Ng |
| B. subtilis | 15 | 35 |
| B. cereus | 20 | 35 |
| C. perfringens | 5 | Ng |
| K. pneumoniae | 2 | Ng |
| L. lactis | 1 | Ng |
| M. smegmatis | 5 | Ng |
| S. marcescens | 3 | Ng |
| S. pyogenes | 3 | Ng |
| A. viridans | 10 | Ng |
| E. aerogenes | 2 | Ng |
| S. liquefaciens | 2 | Ng |
| P. vulgaris | 15 | Ng |
| S. enteridis | 4 | Ng |
| P. mirabilis | 5 | Ng |
| S. abaetetuba | 15 | Ng |
| L. monocytogenes | 20 | Ng |
| N. gonorrhoeae | 5 | Ng |
| Legionella | 10 | 35 |
| M. gordanoae | 14 | 35 |
| M. catarrhalis | 3 | Ng |

“Ng” denotes no growth

Antiviral Testing

The virucidal efficacy of the composition of Example 3 against Coronavirus was tested. The results showed the composition to have an antiviral effect when diluted to 0.25% v/v.

Antiseptic Spray

The composition of Example 1 was formulated into a nitrogen aerosol.

Gangrene

The aerosol formulation was sprayed immediately after debriding and directly onto the wound of an amputated gangrenous limb part. 11 patients have been treated according to this protocol. No re-infection was observed.

Palliative Care—Infected Cancer Tumors

The above aerosol spray has been observed to be effective in controlling infection and completely eradicating offensive odors caused by infected cancer tumors.

Tuberculosis

A trial of 3 patients infected with an antibiotic resistant strain of tuberculosis was conducted. Pulmonary administration of the antiseptic spray formulated to disperse particles of about 10 microns cleared the infection within 10 days of commencing treatment.

Periodontal Applications

The composition of the invention has been observed to successfully control intransigent periodontal infection in 5 patients.

Surgical Masks

The present inventor has observed that surgical masks typically only filter micro-organisms for a period of about 20 minutes after which they become ineffective. After this time, there is very little filtering, if any, of particles including micro-organisms. A mask was sprayed with an aerosol containing the composition from Example 1 in medical grade alcohol as a solvent. The aerosol used nitrogen as the propellent and the particle size of the dispersed droplets was about 10 micron. The mask was sprayed at a rate of ½ g per second on both sides. The concentration of active compounds in the spray was 6,000 ppm. The sprayed mask was observed to increase the effective lifetime of the surgical mask from 20 minutes until up to about 3½ hours.

### Example 4

A second composition of the present invention was prepared from Melaleuca alternifolia as per Example 1. The composition of the oil is as follows:

| | |
|---|---|
| terpen-4-ol | 60-62% |
| alpha terpineol | 4.5-6.5% |
| aromadendrene | 2.0-4.0% |
| viridiflorene | 1.5-3.0% |
| delta cadinene | 1.5-3.5% |
| globulol | 0.5-2.0% |
| viridflorol | 0.3-0.8% |
| monoterpenes | bdl-10% |

Dermal Toxicity Studies

Thirty Duncan-Hartley Guinea Pigs (25 females and 5 males) were ranked by weight and sex and then randomly allocated into five groups of females (n=5 per group) and one group of males (n=5). The treatment groups met the regulatory requirements of having the No Observable Effects Limit (“NOEL”) group, the Lowest Observable Effects Limit (“LOEL”) group, and the Mean Dose Lethality (MDL) or Maximum Toxicity Dose (“MTD”) group.

On Day 0 of the study, Groups 1 (NOEL) and 3 (MTD/MDL) were treated with a 5%, 15%, and 25% of the composition of Example 4 in vitamin E. Group 4 received treatment with 100% base composition and served as an extreme positive control. Group 5 received the vehicle, consisting of a specifically Vitamin E base and served as a primary negative control. Treatment was administered via a treated gauze pad containing 2 ml of the test substance applied directly to a 2 cm square shaved area on the dorsum of each guinea pig. This provided a direct application of 1 ml/cm². The five male guinea pigs were additional to Group 3 and met the requirement of having on test a group of alternate sex subjects receiving the MTD/MDL dose.

Health scores were documented for each individual guinea pig twice daily. Observations were more frequent on the day of treatment. Particular attention was paid to immunogenic, vasodilatory or necrotizing changes of the treatment area. Post mortem examinations were performed on all animals at the end of the study, as none had died on test and any gross abnormalities were documented on a Necropsy Record. Histological examination was conducted on skin tissues taken from the treatment area from each group. Had any gross abnormalities been observed on necropsy, these tissues would have been submitted for histopathological examination. There were no abnormalities identified and therefore this procedure was not performed.

Abnormal findings occurred in those animals on test designated as Group 4 or also identified as the positive control group. It was anticipated that necrotizing changes would occur within this group. None of the animals in the test dose groups had been affected and therefore it was determined that there was no toxic effect when administered cutaneously to guinea pigs at the 5%, 15% and 25% levels as an acute study for toxicity. Gross pathology, and health scores were basically normal for the animals on test. Only those animals in the positive control group demonstrated any abnormal findings as was anticipated. There was no evidence of an induced response between those animals receiving the test substances and those receiving the negative control substance resulting in an identifiable or measurable difference of any significance. Weight gain analysis demonstrated a strong equivalence amongst all groups over the duration of the test including the positive control group suggesting that any adverse changes were local and not systemic in nature. The overall finding of the study was that the base composition, when administered as a single bolus to guinea pigs on an acute toxicity test by dermal application, did not demonstrate any increasing toxic effect corresponding to dosage.

The absence of toxicity even at the highest test dosage of a 25% solution is suggestive that the anticipated human exposure to a 5% solution would have no adverse effect.

This result may be compared to commercially available Melaleuca oil where contact dermatitis is recognized as a potential adverse side affect.

Microbiological Testing

1. Methicillin-Resistant Staphylococcus aureus

A study was conducted to test a solution of the composition of Example 4 on a Methicillin-resistant strain of Staphylococcus aureus using the agar dilution technique as discussed above.

Test Organisms

24 non-replicate isolates from individual patients of Methicillin resistant Staphylococcus aureus ATCC 33591 (mMRSA) were tested.

Plate Preparation

A 10% water soluble stock solution of the composition of Example 4 was added to sterile Mueller Hinton (MH) agar as follows:

| | |
|---|---|
| 0.125% | 0.25 ml stock solution to 20 ml MH agar |
| 0.25% | 0.5 ml stock solution to 20 ml MH agar |
| 0.5% | 1 ml stock solution to 20 ml MH agar |
| 1% | 2 ml stock solution to 20 ml MH agar |
| 2% | 4 ml stock solution to 20 ml MH agar |

The results are shown in the following Table 6

TABLE 6

| Well No. | Control | 0.125% | 0.25% | 0.5% | 1% | 2% |
|---|---|---|---|---|---|---|
| 1 | G | G | NG | NG | NG | NG |
| 2 | G | G | NG | NG | NG | NG |
| 3 | G | NG | NG | NG | NG | NG |
| 4 | G | G | NG | NG | NG | NG |
| 5 | G | G | NG | NG | NG | NG |
| 6 | G | G | NG | NG | NG | NG |
| 6 | G | G | NG | NG | NG | NG |
| 7 | G | G | NG | NG | NG | NG |
| 8 | G | NG | NG | NG | NG | NG |
| 9 | G | G | NG | NG | NG | NG |
| 10 | G | NG | NG | NG | NG | NG |
| 11 | G | NG | NG | NG | NG | NG |
| 12 | G | NG | NG | NG | NG | NG |
| 13 | G | NG | NG | NG | NG | NG |
| 14 | G | NG | NG | NG | NG | NG |
| 15 | G | NG | NG | NG | NG | NG |
| 16 | G | NG | NG | NG | NG | NG |
| 17 | G | G | NG | NG | NG | NG |
| 18 | G | NG | NG | NG | NG | NG |
| 19 | G | G | NG | NG | NG | NG |
| 20 | G | NG | NG | NG | NG | NG |
| 21 | G | NG | NG | NG | NG | NG |
| 22 | G | NG | NG | NG | NG | NG |
| 23 | G | G | NG | NG | NG | NG |
| 24 | G | NG | NG | NG | NG | NG |
| 25 | G | G | NG | NG | NG | NG |
| 26 | G | SLG | NG | NG | NG | NG |

Well No. 1-24 contain clinical isolates of mMRSA
Well No. 25 contains ATCC 33591 as a control
Well No. 26 contains ATCC 25923 as a control
“G” indicates growth or organisms;
“SG” indicates scant growth of organisms;
“NG” indicates no growth of organisms

The results show that all clinical isolates and ATCC control strains of Staphylococcus aureus were inhibited at a concentration of 0.25% v/v.

2. Vancomycin Resistant Enterococcus species

13 vancomycin strains of Enterococcus faecium and 7 vancomycin resistant strains of Enterococcus faecalis were tested against a composition as described in Example 2. Organisms were selected from a time period spanning 5 years to minimize the potential for testing of clonal isolates. 14 strains exhibit the vanB genotype (high level vancomycin resistance plus teicoplanin susceptibility) were tested compared to 6 strains with the vanA genotype (high level vancomycin and teicoplanin resistance). This distribution reflects the predominance of the vanB Enterococcus faecium in nosocomial outbreaks in Australia.

A composition according to Example 4 in a 10% water soluble solution was tested using the agar dilution technique as described above. The composition was tested at dilutions in agar of 0.125%, 0.25%, 0.5%, 1%, 2% and 4%. The results are shown in the following Table 7.

TABLE 7

| Well no. | Control | 0.125% | 0.25% | 0.5% | 1% | 2% | 4% |
|---|---|---|---|---|---|---|---|
| 1 | G | G | G | G | NG | NG | NG |
| 2 | G | G | G | G | NG | NG | NG |
| 3 | G | G | G | G | NG | NG | NG |
| 4 | G | G | G | G | NG | NG | NG |
| 5 | G | G | G | G | NG | NG | NG |
| 6 | G | G | G | G | NG | NG | NG |
| 7 | G | G | G | G | NG | NG | NG |
| 8 | G | G | G | G | NG | NG | NG |
| 9 | G | G | G | G | NG | NG | NG |
| 10 | G | G | G | G | NG | NG | NG |
| 11 | G | G | G | G | NG | NG | NG |
| 12 | G | G | G | G | NG | NG | NG |
| 13 | G | G | G | G | NG | NG | NG |
| 14 | G | G | G | G | NG | NG | NG |
| 15 | G | G | G | G | NG | NG | NG |
| 16 | G | G | G | G | NG | NG | NG |
| 17 | G | G | G | G | NG | NG | NG |
| 18 | G | G | G | G | NG | NG | NG |
| 19 | G | G | G | G | NG | NG | NG |
| 20 | G | G | G | G | NG | NG | NG |
| 21 | G | G | G | G | NG | NG | NG |
| 22 | G | G | G | G | NG | NG | NG |

Wells 1, 3, 6 and 8 contain E. faecium vanA genotype
Wells 2, 5, 9, 16 and 20 contain E. faecalis vanB genotype.
Wells 4, 7, 10, 12, 14, 15, 17, 18 and 19 contain E. faecium vanB genotype.
Wells 11 and 13 contain E. faecalis vanA genotype.
Well 21 contains Control ATC 29212.
Well 22 contains control ATCC 51299

The results show that all clinical isolates and ATT control strains of vancomycin resistant Enterococcus were inhibited at a concentration of 1% v/v irrespective of genotype and species.

3. Clinical Isolates of ESBL and MRA

Clinical isolates of Extended Spectrum Beta-Lactamase (ESBL) producing Gram Negative organisms and multi-resistant Acinetobacter baumanni (MRA) were tested against a 10% water soluble solution of the composition of Example 4 using the agar dilution technique as described above.

28 isolates with ESBL and 14 strains of MRA were tested. Organisms were selected from a time period spanning 3 years to minimize the potential for testing of clonal isolates. A selection of different species with ESBLs was tested including Escherichia coli.

A composition according to Example 4 in a 10% water soluble solution was tested using the agar dilution technique as described above. The composition was tested at dilutions in agar of 0.125%, 0.25%, 0.5%, 1%, 2% and 4%. The results are shown in the following Tables 8 and 9:

TABLE 8

| Well no. | Control | 0.125% | 0.25% | 0.5% | 1% | 2% | 4% |
|---|---|---|---|---|---|---|---|
| 1 | G | G | NG | NG | NG | NG | NG |
| 2 | G | G | G | NG | NG | NG | NG |
| 3 | G | G | NG | NG | NG | NG | NG |
| 4 | G | G | NG | NG | NG | NG | NG |
| 5 | G | G | NG | NG | NG | NG | NG |
| 6 | G | G | G | NG | NG | NG | NG |
| 7 | G | G | G | NG | NG | NG | NG |
| 8 | G | G | G | NG | NG | NG | NG |
| 9 | G | G | G | NG | NG | NG | NG |
| 10 | G | G | G | NG | NG | NG | NG |
| 11 | G | G | G | NG | NG | NG | NG |
| 12 | G | G | NG | NG | NG | NG | NG |
| 13 | G | G | G | NG | NG | NG | NG |
| 14 | G | G | G | NG | NG | NG | NG |
| 15 | G | G | G | NG | NG | NG | NG |
| 16 | G | G | G | NG | NG | NG | NG |
| 17 | G | G | G | NG | NG | NG | NG |
| 18 | G | G | G | NG | NG | NG | NG |
| 19 | G | G | G | NG | NG | NG | NG |
| 20 | G | G | G | NG | NG | NG | NG |
| 21 | G | G | G | NG | NG | NG | NG |
| 22 | G | G | G | NG | NG | NG | NG |

Wells 1 to 14 contain strains of Acinetobacter baumanni.
Well 15 contains Escherichia coli.
Wells 16 to 19 contain strains of Klebseiella pneumonia.
Well 20 contains Enterobacter cloacae.
Well 21 contains ATCC 25923 as a control.

TABLE 9

| Well No. | Control | 0.125% | 0.25% | 0.5% | 1% | 2% | 4% |
|---|---|---|---|---|---|---|---|
| 1 | G | G | G | NG | NG | NG | NG |
| 2 | G | G | G | NG | NG | NG | NG |
| 3 | G | G | G | NG | NG | NG | NG |
| 4 | G | G | G | NG | NG | NG | NG |
| 5 | G | G | G | NG | NG | NG | NG |
| 6 | G | G | G | NG | NG | NG | NG |
| 7 | G | G | G | NG | NG | NG | NG |
| 8 | G | G | G | NG | NG | NG | NG |
| 9 | G | G | G | NG | NG | NG | NG |
| 10 | G | G | G | NG | NG | NG | NG |
| 11 | G | G | G | NG | NG | NG | NG |
| 12 | G | G | NG | NG | NG | NG | NG |
| 13 | G | G | G | NG | NG | NG | NG |
| 14 | G | G | G | NG | NG | NG | NG |
| 15 | G | G | G | NG | NG | NG | NG |
| 16 | G | G | G | NG | NG | NG | NG |
| 17 | G | G | NG | NG | NG | NG | NG |
| 18 | G | G | G | NG | NG | NG | NG |
| 19 | G | G | G | NG | NG | NG | NG |
| 20 | G | G | G | NG | NG | NG | NG |
| 21 | G | G | G | NG | NG | NG | NG |
| 22 | G | G | G | NG | NG | NG | NG |

Well Nos. 1 to 4, 11, 12, 16, 17, 19 and 21 contain Escherichia coli.
Well Nos. 5, 13, 15, 18 and 20 contain Klebsiella pneumoniae
Well Nos. 6-8, 16 and 22 contain Enterobacter cloacae.

The results show that complete inhibition of all clinical isolates with ESBL and Acinetobacter baumanni was achieved at a concentration of the inventive composition of 0.5% v/v.

4. Anti-Tuberculosis Activity

Mycobacterium tuberculosis ATCC 27294 and Mycobacterium smegmatis ATCC 14468 was tested with a composition as described in Example 4.

Inocula Preparation

Loopfuls of cells from working cultures on Middlebrook 7H11 agar were transferred into 15 ml of Middlebrook 7H9 broth and then placed in a 100 ml flask with glass beads. The flask was shaken for 3 min using a mechanical shaker. The suspensions were aspirated from the glass beads and transferred into another tube. The number of cells in suspensions was adjusted to ca 0.5 McFarland standard turbidity with 7H9. The working suspensions were prepared in a 1:5 dilution with saline and used within 2 h.

Modified EN 1276 Method

The European Standard EN 1276, incorporated by reference, modified to overcome potential problems with loss of activity dues to volatility of actives in the composition was used to evaluate various dilutions of the composition of Example 4.

Three ml of various concentrations of the composition of Example 4 were mixed with 3 ml of bacterial suspensions and left for various times of exposure (5-60 min). After exposure, 1 ml aliquots of the mixture were taken out and added to a tube containing 8 ml of neutralizer and 1 ml of SDW and mixed. After 5 min neutralization time, a 0.5 aliquot was removed for inoculation into a MGIT tube. This was placed on the Bactec 960 and monitored for growth of the mycobacterium for 4 weeks. For positive controls, the composition was replaced with saline and exposed for 60 minutes. For negative controls, a solution of the 10% composition (2%) which in a preliminary trial inhibited M. tuberculosus ATCC 27294 and M. smegmatis ATCC 14468, was mixed with bacterial suspensions and left for 60 min.

Test Composition

A solution of Example 4 at 10% containing 1% vitamin E was prepared using distilled water to concentrations between 0.03-4%.

The results are shown in the following Tables, which show the time to positive at various concentrations of a 10% solution of Example 4:

| | | | | | | | | | |
|---|---|---|---|---|---|---|---|---|---|
| Exposure | | | | | | | | | |
| time | 0.03 | 0.06 | 0.125 | 0.25 | 0.5 | 1.0 | 2.0 | +ve | −ve |
| (min) | (h) | (h) | (h) | (h) | (h) | (h) | (h) | (h) | (h) |

M. tuberculosis

| | | | | | | | |
|---|---|---|---|---|---|---|---|
| 5 | 340 | 334 | 366 | NG | NG | | |
| 15 | 381 | 357 | 398 | NG | NG | | |
| 30 | 419 | 380 | 424 | NG | NG | | |
| 60 | 314 | 443 | NG | NG | NG | 366 | NG |

M. smegmatis

| | | | | | | |
|---|---|---|---|---|---|---|
| 5 | 63 | 62 | 75 | 101 | | |
| 15 | 58 | 67 | 87 | NG | | |
| 30 | 62 | 67 | 95 | NG | | |
| 60 | 63 | 67 | 136 | NG | 60 | NG |

The results show that the MIC for the composition of Example 4 was 0.125% after bacterial cells were exposed to the composition for 60 minutes using the BACTEC Mycobacteria Growth Indicator Tube system (BACTEC 960/MGIT). The result was validated using the rapid grower M. smegmatis. For M. smegmatis, the MIC for a 10% solution of the composition of Example 4 was 1% after the cells were exposed for 15 minutes. The results indicate that the Mycobacterium tuberculosis ATCC 27294 is susceptible to a solution of the inventive composition at a concentration of 0.125%

5. Avian Influenza

### H5N1 Avian Influenza Virus

This work was done to confirm the ability of the composition of Example 4 to act as a virucidal agent against a Vietnamese H5N1 highly pathogenic avian influenza virus strain. The trials were done in specific pathogen free chicken eggs, these being the most sensitive culture system for avian influenza viruses. The procedure was modified from ASTM E1052-96 “Standard Test Method for Efficacy of Antimicrobial Agents against Viruses in Suspension”, incorporated by reference.

Material and Methods

A 10% solution of the composition of Example 4 was made in dimethyl sulphoxide (DMSO) by adding 1.0 ml of the composition to 9.0 ml of DMSO and mixing thoroughly. A solution of 1% with no emulsifiers was used as supplied for virus treatment.

Virus

The virus used for this work was A/chicken/Vietname/8/2004 H5N1 grown in the allantoic sac of embryonated, SPF chicken eggs. Infectious allantoic fluid was harvested, pooled and stored at −80° C. for this trial. The microstores pool reference number of this material is 0404-30-1550.

Trial 3—60, 120 and 240 Minute Contact Time with Virus Titrated in SPF Eggs

Virus Treatment

The 10% solution of Example 4 was diluted 1:5, 1:3.3 and 1:2.5 in phosphate buffered saline (pH 7.3) to give final concentrations of 2%, 3% and 4% respectively. Based on ASTM E1052-96, 0.1 ml of virus was mixed with 0.4 ml of the composition of Example 2 at concentrations of 2%, 3% and 4%. Mock-treated virus, consisting of 0.1 ml of virus mixed with 0.4 ml of a 1:5 dilution of DMSO without any inventive composition in PBS and untreated virus, consisting of 0.1 ml of virus mixed with 0.4 ml of PBS, were also prepared. All mixtures were incubated at room temperature for the respective times. 2%, 3% and 4% of the inventive composition were diluted 1:10 in PBS and 0.1 ml inoculated into 5 eggs each.

Virus Titrations

Trial 2—in SPF Eggs

Residual virus was assayed by making 10-fold dilutions in PBS of each virus mixture from 10⁻¹ to 10⁻⁸. 0.2 ml of virus mixture was added to 1.8 ml of PBS and mixed thoroughly to give a 10⁻¹ dilution. 0.2 ml of this was added to 1.8 ml of PBS and mixed thoroughly to give a 10⁻² dilution and so on to a final dilution of 10⁻⁸. 0.1 ml of each dilution was inoculated into the allantoic sac of embryonated, SPF chicken eggs, incubated at 37° C. for 3 days or until embryo death. Eggs were examined twice daily for viability. At death or after 3 days incubation all eggs were chilled overnight at 4° C. and then tested for the presence of haemagglutination and an indicator of virus infection. The residual infectivity titre was calculated by the method of Reed and Meunch.

Results

Eggs inoculated with the inventive composition alone showed no egg deaths, showing that mortalities were due to the effects of the virus inoculum.

Untreated Virus

| | Time post-inoculation | | | | | | |
|---|---|---|---|---|---|---|---|
| | | | | | 9/6 | 9/6 | HA |
| Dilution | 7/6 am | 7/6 pm | 8/6 am | 8/6 pm | am | pm | Result |
| 10⁻¹ | — | 5 | | | | | 5/5 positive |
| 10⁻² | — | — | 5 | | | | 5/5 positive |
| 10⁻³ | — | — | 5 | | | | 5/5 positive |
| 10⁻⁴ | — | — | 2 | 3 | | | 5/5 positive |
| 10⁻⁵ | — | — | — | 5 | | | 5/5 positive |
| 10⁻⁶ | — | — | — | 5 | — | — | 5/5 positive |
| 10⁻⁷ | — | — | — | 3 | — | — | 3/5 positive |
| 10⁻⁸ | — | — | — | — | — | — | 5/5 negative |

Virus titre = 10^{7.1} egg infectious doses per 0.1 ml

Mock-Treated Virus

| | Time post-inoculation | | | | | | |
|---|---|---|---|---|---|---|---|
| | | | | | 9/6 | 9/6 | |
| Dilution | 7/6 am | 7/6 pm | 8/6 am | 8/6 pm | am | pm | HA Result |
| 10⁻¹ | — | 5 | | | | | 5/5 positive |
| 10⁻² | — | — | 5 | | | | 5/5 positive |
| 10⁻³ | — | — | 5 | | | | 5/5 positive |
| 10⁻⁴ | — | — | 2 | 3 | | | 5/5 positive |
| 10⁻⁵ | — | — | 3 | 2 | | | 5/5 positive |
| 10⁻⁶ | 2 | — | 3 | — | | | 5/5 positive |
| 10⁻⁷ | — | — | 4 | — | — | — | 4/5 positive |
| 10⁻⁸ | — | — | 1 | — | — | — | 1/5 positive |

Virus titre = 10^{7.5} egg infectious doses per 0.1 ml

2% Treated Virus

60 Minute Contact Time

| | Time post-inoculation | | | | | | |
|---|---|---|---|---|---|---|---|
| | | | | | 4/8 | 4/8 | |
| Dilution | 2/8 am | 2/8 pm | 3/8 am | 3/8 pm | am | pm | HA Result |
| 10⁻¹ | — | 5 | | | | | 5/5 positive |
| 10⁻² | — | — | 5 | | | | 5/5 positive |
| 10⁻³ | — | — | 5 | | | | 5/5 positive |
| 10⁻⁴ | — | — | 3 | — | | | 3/5 positive |
| 10⁻⁵ | — | — | — | — | | | 0/5 positive |
| 10⁻⁶ | — | — | — | — | — | — | 0/5 positive |
| 10⁻⁷ | — | — | — | — | — | — | 0/5 positive |
| 10⁻⁸ | — | — | — | — | — | — | 0/5 negative |

Virus titre = 10^{4.1} egg infectious doses per 0.1 ml

120 Minute Contact Time

| | Time post-inoculation | | | | | | |
|---|---|---|---|---|---|---|---|
| | | | | 21/8 | 22/8 | 22/8 | |
| Dilution | 20/8 am | 20/8 pm | 21/8 am | pm | am | pm | HA Result |
| 10⁻¹ | — | 5 | | | | | 5/5 positive |
| 10⁻² | — | — | 5 | | | | 5/5 positive |
| 10⁻³ | — | — | 5 | | | | 5/5 positive |
| 10⁻⁴ | — | — | 4 | — | — | — | 2/5 positive |
| 10⁻⁵ | — | — | 1 | — | — | — | 0/5 positive |
| 10⁻⁶ | — | — | — | — | — | — | 0/5 positive |
| 10⁻⁷ | — | — | — | — | — | — | 0/5 positive |
| 10⁻⁸ | — | — | — | — | — | — | 0/5 positive |

Virus titre = 10^{4.5} egg infectious doses per 0.1 ml

240 Minute Contact Time

| | Time post-inoculation | | | | | | |
|---|---|---|---|---|---|---|---|
| | | | | 28/8 | 29/8 | 29/8 | |
| Dilution | 27/8 am | 27/8 pm | 28/8 am | pm | am | pm | HA Result |
| 10⁻¹ | — | 5 | | | | | 5/5 positive |
| 10⁻² | — | — | 5 | | | | 5/5 positive |
| 10⁻³ | — | — | 3 | — | — | — | 5/5 positive |
| 10⁻⁴ | — | — | — | — | — | | 3/5 positive |
| 10⁻⁵ | — | — | — | — | — | — | 0/5 positive |
| 10⁻⁶ | — | — | — | — | — | — | 0/5 positive |
| 10⁻⁷ | — | — | — | — | — | — | 0/5 positive |
| 10⁻⁸ | — | — | — | — | — | — | 0/5 positive |

Virus titre = 10^{3.1} egg infectious doses per 0.1 ml

3% Treated Virus

60 Minute Contact Time

| | Time post-inoculation | | | | | | |
|---|---|---|---|---|---|---|---|
| | | | | | 4/8 | 4/8 | |
| Dilution | 2/8 am | 2/8 pm | 3/8 am | 3/8 pm | am | pm | HA Result |
| 10⁻¹ | — | 5 | | | | | 5/5 positive |
| 10⁻² | — | — | 5 | | | | 5/5 positive |
| 10⁻³ | — | — | 2 | — | — | — | 2/5 positive |
| 10⁻⁴ | — | — | — | — | — | — | 0/5 positive |
| 10⁻⁵ | — | — | — | — | — | — | 0/5 positive |
| 10⁻⁶ | — | — | — | — | — | — | 0/5 positive |
| 10⁻⁷ | — | — | — | — | — | — | 0/5 positive |
| 10⁻⁸ | — | — | — | — | — | — | 0/5 negative |

Virus titre = 10^{2.9} egg infectious doses per 0.1 ml

120 Minute Contact Time

| | Time post-inoculation | | | | | | |
|---|---|---|---|---|---|---|---|
| | | | | 21/8 | 22/8 | 22/8 | |
| Dilution | 20/8 am | 20/8 pm | 21/8 am | pm | am | pm | HA Result |
| 10⁻¹ | — | 5 | | | | | 5/5 positive |
| 10⁻² | — | — | 5 | | | | 5/5 positive |
| 10⁻³ | — | — | 4 | — | — | — | 5/5 positive |
| 10⁻⁴ | — | — | 1 | — | — | — | 1/5 positive |
| 10⁻⁵ | — | — | — | — | — | — | 0/5 positive |
| 10⁻⁶ | — | — | — | — | — | — | 0/5 positive |
| 10⁻⁷ | — | — | — | — | — | — | 0/5 positive |
| 10⁻⁸ | — | — | — | — | — | — | 0/5 positive |

Virus titre = 10^{3.5} egg infectious doses per 0.1 ml

240 Minute Contact Time

| | Time post-inoculation | | | | | | |
|---|---|---|---|---|---|---|---|
| | | | | 28/8 | 29/8 | 29/8 | |
| Dilution | 27/8 am | 27/8 pm | 28/8 am | pm | am | pm | HA Result |
| 10⁻¹ | — | 5 | | | | | 5/5 positive |
| 10⁻² | — | — | 5 | | | | 5/5 positive |
| 10⁻³ | — | — | 2 | — | — | — | 2/5 positive |
| 10⁻⁴ | — | — | — | — | — | | 0/5 positive |
| 10⁻⁵ | — | — | — | — | — | — | 0/5 positive |
| 10⁻⁶ | — | — | — | — | — | — | 0/5 positive |
| 10⁻⁷ | — | — | — | — | — | — | 0/5 positive |
| 10⁻⁸ | — | — | — | — | — | — | 0/5 positive |

Virus titre = 10^{2.9} egg infectious doses per 0.1 ml

4% Treated Virus

60 Minute Contact Time

| | Time post-inoculation | | | | | | |
|---|---|---|---|---|---|---|---|
| | | | | 14/8 | 15/8 | 15/8 | |
| Dilution | 13/8 am | 13/8 pm | 14/8 am | pm | am | pm | HA Result |
| 10⁻¹ | — | 5 | | | | | 5/5 positive |
| 10⁻² | — | 5 | | | | | 5/5 positive |
| 10⁻³ | — | 3 | — | — | — | — | 3/5 positive |
| 10⁻⁴ | — | — | — | — | — | — | 0/5 positive |
| 10⁻⁵ | — | — | — | — | | | 0/5 positive |
| 10⁻⁶ | — | — | — | — | — | — | 0/5 positive |
| 10⁻⁷ | — | — | — | — | — | — | 0/5 positive |
| 10⁻⁸ | — | — | — | — | — | — | 0/5 negative |

Virus titre = 10^{3.1} egg infectious doses per 0.1 ml

120 Minute Contact Time

| | Time post-inoculation | | | | | | |
|---|---|---|---|---|---|---|---|
| | | | | 21/8 | 22/8 | 22/8 | |
| Dilution | 20/8 am | 20/8 pm | 21/8 am | pm | am | pm | HA Result |
| 10⁻¹ | — | 5 | | | | | 5/5 positive |
| 10⁻² | — | — | 5 | | | | 5/5 positive |
| 10⁻³ | — | — | 1 | — | — | — | 1/5 positive |
| 10⁻⁴ | — | — | — | — | — | — | 0/5 positive |
| 10⁻⁵ | — | — | — | — | — | — | 0/5 positive |
| 10⁻⁶ | — | — | — | — | — | — | 0/5 positive |
| 10⁻⁷ | — | — | — | — | — | — | 0/5 positive |
| 10⁻⁸ | — | — | — | — | — | — | 0/5 positive |

Virus titre = 10^{2.7} egg infectious doses per 0.1 ml

240 Minute Contact Time

| | Time post-inoculation | | | | | | |
|---|---|---|---|---|---|---|---|
| | | | | | 5/9 | 5/9 | |
| Dilution | 3/9 am | 30/8 pm | 4/8 am | 31/8 pm | am | pm | HA Result |
| 10⁻¹ | — | — | — | — | 1 | — | 0/5 positive |
| 10⁻² | — | — | — | — | — | — | 0/5 positive |
| 10⁻³ | — | — | — | — | — | — | 0/5 positive |
| 10⁻⁴ | — | — | — | — | — | — | 0/5 positive |
| 10⁻⁵ | — | — | — | — | — | — | 0/5 positive |
| 10⁻⁶ | | | | | | | |
| 10⁻⁷ | | | | | | | |
| 10⁻⁸ | | | | | | | |

Virus titre = 10 egg infectious doses per 0.1 ml

### Summary of Titration Results of Trial 3

### Log 10 Residual Virus Titre After Treatment in (EID ₅₀/0.1 ml)

| | Treatment Time | | |
|---|---|---|---|
| concentration | 60 Minutes | 120 Minutes | 240 Minutes |
| 2% | 4.1 | 4.5 | 3.1 |
| 3% | 2.9 | 3.5 | 2.9 |
| 4% | 3.1 | 2.7 | 0 |
| Untreated virus | 7.1 | | |
| Mock-treated virus | 7.5 | | |

Eggs inoculated with 1:10 dilutions of the inventive solutions alone showed no egg deaths, showing that mortalities were due to the effects of the virus inoculum.

It may be appreciated that the extract composition of the present invention exhibits negligible levels of toxicity when compared to conventional Melaleuca essential oil which contains significant amounts of monoterpenes. This enables the composition to be used in a wide range of applications for which conventional Melaleuca oil would be unsuitable. A particular advantage of the reduction in toxicity is the ability to use the composition of the present invention in medical applications.

Despite the dramatic reduction in monoterpene content and change in overall phytochemical profile, the extract or composition of the present invention has not only retained but improved its efficacy.

It will be appreciated that various changes or modifications may be made to the invention as described and claimed herein without departing from the spirit and scope thereof.

1. A composition producable by removing at least 80% of the total monoterpene content from a conventional Melaleuca oil that conforms to Australian standard AS 2782-1997 or to International standard ISO 4730:2004;
wherein said composition contains 10% or less monoterpenes; and
wherein said monoterpenes have the formula C₁₀H₁₅.

2. The composition of claim 1, wherein the conventional Melaleuca oil is obtained from Melaleuca alternifolia.

3. The composition of claim 1, wherein the conventional Melaleuca oil is extracted from Melaleuca plants by steam distillation.

4. The composition of claim 1, wherein the conventional Melaleuca oil conforms to the Australian standard.

5. The composition of claim 1, wherein the conventional Melaleuca oil conforms to the International standard.

6. The composition of claim 1, which contains 5% or less total monoterpene content.

7. The composition of claim 1, which contains 2.5% or less total monoterpene content.

8. The composition of claim 1, wherein at least 80% of the total monoterpene content is removed under reduced pressure (less than 1 atm) at a temperature of less than 50° C.

9. The composition of claim 1, wherein at least 80% of at least one monoterpene selected from the group consisting of ∝-pinene, ∝-thujene, β-pinene, sabinene, ∝-phellandrene, ∝-terpinene, limonene, γ-terpinene, β-phellandrene, γ-terpinene and terpinolene is removed.

10. The composition of claim 1 which consists essentially of a conventional Melaleuca alternifolia oil as defined by the Australian or International standard from which at least 80% of the monoterpene content has been removed providing a total monoterpene content of 10% or less.

11. The composition of claim 10 which contains between about 50% to about 70% terpenin-4-ol.

12. The composition of claim 10 which contains between about 60% to about 65% terpenin-4-ol.

13. The composition of claim 10 which contains:

| | |
|---|---|
| terpenin-4-ol | 52-62% |
| ∝-terpineol | 4-6.5% |
| aromadendrene | 2-7% |
| viridiflorene | 1.5-5% |
| δ-cadinene | 1.5-3.5% |
| globulol | 0.5-2% and |
| viridiflorol | 0.3-0.8%; or |
| terpinene-4-ol | 52-54% |
| ∝-terpineol | 4-6% |
| aromadendrene | 5-7% |
| viridiflorene | 3-5% |
| δ-cadinene | 1.5-3.5% |
| globulol | 1-2% and |
| viridflorol | 0.3-0.6%. |

14. The composition of claim 1, further comprising at least one least one lipophilic or hydrophobic carrier, aqueous carrier, diluent, emulsifier, soap, detergent, antiseptic, antioxidant, oxidant, excipient, or solvent.

15. A bandage, wound dressing, or patch; a composition formulated for topical application, containing the composition of claim 1.

16. An aerosol comprising the composition of claim 1 dispersed in a propellant.

17. The aerosol of claim 17 that has a particle size ranging from 5 to 20 microns.

18. The aerosol of claim 16 that has a particle size ranging from 40 to 60 microns.

19. A composition that contains 10% or less monoterpenes having the formula C₁₀H₁₆ which comprises:

| | |
|---|---|
| terpenin-4-ol | 52-66% |
| ∝-terpineol | 4-6.5% |
| aromadendrene | 1-7% |
| viridiflorene | 0.5-5% |
| δ-cadinene | 1.5-3.5% |
| globulol | 0.3-2% and |
| viridiflorol | 0.1-0.8%. |

20. The composition of claim 19 that contains less than 5% monoterpenes.
